(19) United States
(12) Patent Application Publication
(10) Pub. No.: US 2022/0375327 A1
(43) Pub. Date: Nov. 24, 2022

(54) SENSOR DEVICE FOR DETECTING DISINFECTING STATE

(71) Applicant: MPICS INNOVATIONS PTE. LTD, Singapore (SG)

(72) Inventors: Kok Keong Richard LUM, Singapore (SG); Hong Sia TAN, Singapore (SG)

(21) Appl. No.: 17/839,662

(22) Filed: Jun. 14, 2022

Related U.S. Application Data
(63) Continuation-in-part of application No. 17/375,881, filed on Jul. 14, 2021.
Application No. 17/375,881 is a continuation of application No. 17/328,710, filed on May 24, 2021.

(51) Int. Cl. G08B 21/24; G01J 1/04; G01J 1/02; H04W 52/02; G01J 1/44; G01J 1/50

(52) U.S. Cl. CPC G08B 21/245; G01J 1/0407; G01J 1/0271; H04W 52/0235; G01J 1/44; G01J 1/50; G01J 2001/446

(57) ABSTRACT

Devices and methods for detecting a disinfecting state are described. An example of a sensor device is disclosed to include: a housing; a radiation sensitive material disposed on one or more portions of an external surface of the housing; a sensor configured to measure intensity information associated with ultraviolet (UV) radiation of a first frequency band; a controller configured to record the intensity information, temporal information associated with measuring the intensity information, or both; and a transceiver device configured to transmit and receive radio frequency (RF) signals.

### CROSS-REFERENCE TO RELATED APPLICATION

The present application is a continuation-in-part of U.S. patent application Ser. No. 17/375,881, filed on Jul. 14, 2021, which is a continuation of U.S. patent application Ser. No. 17/328,710, filed May 24, 2021, the entire disclosure of which is hereby incorporated by reference, in its entirety, for all that it teaches and for all purposes.

### FIELD OF TECHNOLOGY

The following relates to disinfection, including the disinfection of pathogens using ultraviolet (UV) light and UV sensors.

### BACKGROUND

Some cleaning techniques utilize ultraviolet (UV) radiation to inactivate bacteria and viruses from a physical environment, for example, from surfaces in the physical environment. In some cases, the amount of UV radiation energy a surface is exposed to during a disinfecting procedure may contribute to whether the surface is fully sanitized. For example, factors such as the radiant power of UV light applied during a disinfecting procedure, along with the duration over which the UV light is applied may determine whether the surface has been fully sanitized. Disinfection techniques are desired which may achieve a target effectiveness while minimizing related overhead (e.g., time, power, etc.).

### SUMMARY

The described techniques relate to improved methods, systems, devices, and apparatuses that support an ultraviolet (UV) disinfection system with sensors and feedback. The described techniques further relate to improved methods, systems, devices, and apparatuses that support a sensor device for detecting a disinfecting a state.

In one aspect, a system is provided that includes: an enclosure; a radiation source configured to emit UV radiation located within the enclosure towards a targeted area; a holding area located within the targeted area configured to receive the UV radiation; and one or more sensor devices, the one or more sensor devices comprising a sensor configured to detect the UV radiation having a UV profile. In some aspects, the one or more sensor devices are configured to record intensity information associated with the UV radiation. In some aspects, one of the radiation source and the holding area is fixed within the enclosure and the other one of the radiation source and the holding area is movable so as to adjust an intensity of the UV radiation received at the one or more sensor devices by way of controlling a distance between the holding area and the radiation source.

In some aspects, the one or more sensor devices further comprise an additional sensor configured to detect the UV radiation having an additional UV profile. In some aspects, the UV profile is wider than the additional UV profile.

In some aspects, the UV profile allows a first radiation with wavelengths between 220 nanometers (nm) and 400 nm to substantially passthrough, and the additional UV profile allows a second radiation with wavelengths between 300 nm and 400 nm to substantially pass through.

In some aspects, the enclosure includes a compartment of a building or a vehicle.

In some aspects, the radiation source is attached adjacent to a light source located within the enclosure, and the radiation source is attached to a movable structure configured to be lower down from a top surface of the enclosure.

In some aspects, the radiation source is attached to a movable robot configured to approach the targeted area and an additional targeted area of an additional enclosure which are positioned at a fixed location relative to the enclosure and the additional enclosure.

In some aspects, the system further comprises a plurality of additional enclosures. In some aspects, each of the plurality of additional enclosures comprises: an additional radiation source configured to emit additional UV radiation towards an additional targeted area; an additional holding area located within the additional targeted area, the additional holding area configured to receive the additional ultraviolet radiation; one or more additional sensor devices comprising a sensor configured to detect the additional UV radiation and configured to record additional intensity information associated with the additional UV radiation; and a controller configured to communicate with each of the additional radiation source and the one or more additional sensor devices of the plurality of enclosures. In some aspects, the controller is configured to control and maintain an aggregate value of each of the intensity information and the additional intensity information above a predetermined value.

In some aspects, the controller is configured to maintain the aggregate value above the predetermined value by adjusting a time period of the targeted area being exposed to the UV radiation and/or the additional UV radiation of each of the plurality of additional enclosures.

In some aspects, the system further comprises: one or more additional radiation sources configured to emit the UV radiation; one or more additional sensor devices positioned opposing the one or more additional radiation sources within the targeted area, the one or more additional sensor devices configured to record the intensity information associated with the UV radiation; and a controller configured to communicate with each of the additional radiation sources and the one or more additional sensor devices. In some aspects, the controller is configured to control and maintain an aggregate value of the intensity information for each of the one or more sensor devices and the one or more additional sensor devices to be above a predetermined value.

In some aspects, the enclosure has a diameter, and the one or more additional radiation sources are positioned such that a distance between any adjacent two of the one or more additional radiation sources is at least 60% of a length of the diameter.

In another aspect, a system is provided that includes: an enclosure; a radiation source configured to emit UV radiation within the enclosure towards a targeted area; a holding area located within the targeted area configured to receive the UV radiation; and one or more sensor devices, the one or more sensor devices comprising a first sensor configured to detect the UV radiation having a first filter profile and a second sensor configured to detect the UV radiation having a second filter profile. In some aspects, the second filter profile allows a wider band of UV radiation to pass through as compared to the first filter profile. In some aspects, the one or more sensor devices are configured to record intensity information associated with the UV radiation, and a location of one of the radiation source and the holding area is fixed within the enclosure.

In some aspects, the first filter profile and the second filter profile have an overlapping wavelength band within a UV wavelength range of 200 nm and 400 nm.

In some aspects, the first filter profile and the second filter profile have an overlapping wavelength band within a UV wavelength range of 300 nm and 400 nm.

In another aspect, a sensing device is provided that includes: a housing configured to receive an external radiation from a first direction; a first sensor configured to detect an UV radiation having a first filter profile; a first UV filter positioned adjacent to the first sensor within the housing such that a first radiation from the first direction received by the first sensor is configured to pass through the first UV filter; a second sensor configured to detect the UV radiation having a second filter profile, the second filter profile allowing a wider band of UV radiation to pass through as compared to the first filter profile; a second UV filter positioned adjacent to the second sensor within the housing such that a second radiation from the first direction received by the second sensor is configured to pass through the second UV filter; and a controller. In some aspects, the controller is configured to generate and output in accordance with the first radiation received at the first sensor and the second radiation received at the second sensor.

In some aspects, the first UV filter associated with the first filter profile is configured to pass through radiation with wavelengths between 300 nm and 400 nm.

In some aspects, the second UV filter associated with the second filter profile is configured to pass through radiation with wavelengths between 220 nm and 400 nm.

In some aspects, the first filter profile and the second filter profile have an overlapping wavelength band within a UV wavelength range of 200 nm and 400 nm.

In some aspects, the sensing device may further comprise an arithmetic unit configured to perform either a subtraction or an additional function to outputs of the first sensor and the second sensor.

In some aspects, the sensing device may further comprise a control block configured to provide an indication that a wavelength of the UV radiation is inside the overlapping wavelength band.

In some aspects, the sensing device may further comprise a control block configured to provide an indication that a wavelength of the UV radiation is outside the overlapping wavelength band.

In some aspects, the first UV filter and the second UV filter allow radiations of infra-red bandwidths to pass through.

In some aspects, the housing includes an opto-semiconductor package.

In some aspects, the first sensor includes a first die, the second sensor includes a second die, and the controller includes a third die. In some aspects, the first die, the second die, and the third die are located within the opto-semiconductor package.

In some aspects, the sensing device may further comprise a semiconductor chip. In some aspects, the first sensor includes a first photo-sensing area of the semiconductor chip, and the second sensor includes a second photo-sensing area of the semiconductor chip.

In some aspects, the controller includes a logic circuit area of the semiconductor chip.

### BRIEF DESCRIPTION OF THE DRAWINGS

FIG. 1 illustrates an example of a system that supports an ultraviolet (UV) disinfection system with sensors and feedback in accordance with aspects of the present disclosure.

FIG. 2 illustrates an example of a system that supports a UV disinfection system with sensors and feedback in accordance with aspects of the present disclosure.

FIG. 3 illustrates an additional example of a system that supports a UV disinfection system with sensors and feedback in accordance with aspects of the present disclosure.

FIG. 4 illustrates example filter profiles that support a UV disinfection system with sensors and feedback in accordance with aspects of the present disclosure.

FIG. 5 illustrates a system configuration that supports a UV disinfection system with sensors and feedback in accordance with aspects of the present disclosure.

FIGS. 6A and 6B illustrate example housing configurations that support a UV disinfection system with sensors and feedback in accordance with aspects of the present disclosure.

### DETAILED DESCRIPTION

According to example aspects of the present disclosure, an ultraviolet (UV) disinfection system is provided which includes a radiation source and a source device (also referred to herein as a source wireless communication device) coupled to the radiation source. In some aspects, the radiation source may be a UV light source configured to emit light in the UV range. For example, the radiation source may be configured to emit light having a wavelength between 200 nanometers (nm) to 300 nm (i.e., UV-C wavelength range).

The UV disinfection system may include a series of sensor tags (also referred to herein as UV tags, UV sensor tags, or sensor devices). The sensor tags may be, for example, communication devices capable of transmitting and receiving signals (e.g., via wired or wireless communications). In some aspects, each of the sensor tags may include one or more radiation sensors (also referred to herein as light sensors, UV sensors, or electronic UV sensors). In an example, a radiation sensor included in a sensor tag may detect and measure UV radiation (e.g., UV-C radiation).

In some aspects, a radiation sensor of a sensor tag may convert detected UV radiation into digital data (e.g., a digital measurement value), based on which the UV disinfection system may calculate the total UV radiation (e.g., total energy) emitted by the radiation source into the physical environment. In some examples, each sensor tag may communicate measured UV radiation power at the sensor tag (e.g., the total energy of UV radiation). For example, each sensor tag may communicate measured UV radiation power to other devices in the UV disinfection system using radio frequency (RF) communications (e.g., via an RF transceiver included in the sensor tag).

In some examples, a sensor tag may be powered by an electrical power source electrically coupled to the sensor tag. For example, the electrical power source may include an external power source. In another example, the electrical power source may include batteries integrated with the sensor tag (e.g., included in a housing of the sensor tag). The batteries may include long-life or extended-life batteries. In some aspects, the sensor tags (e.g., battery powered, or coupled to an electrical power source) may be attached to any area in a room or a target area. In some examples, the locations of the sensor tags may calculate in consideration of disinfecting the target area (e.g., calculated such that the readings of the sensor tags can be computed to deduce whether the target area is sufficiently disinfected). Sufficient disinfection, for example, may encompass a disinfection level or disinfection coverage (e.g., based on UV emission power, UV emission duration, UV emission coverage) high enough to kill known viruses or germs or meet some standards (e.g., threshold). Power consumption of such sensor tags, for example, may be designed (e.g., usually designed) to be low power consumption.

In some other aspects, the sensor tag may be powered by energy harvesting techniques including solar, piezo-vibrational, or wireless charging. In an example, the sensor tag may be powered by RF wireless waves (e.g., RF wireless charging). For example, the source device may include an RF transceiver, via which the source device may wirelessly power or charge the sensor tag. In some aspects, the UV disinfection system may energize sensor tags (within an RF range of the RF transceiver coupled to the source device) to provide power to enable UV sensing operations of the sensor tags. In some other aspects, the sensor tags may be energized by background RF signals (e.g., Bluetooth, WiFi, 3G, 4G, or 5G sources generated by other communications devices).

In some aspects, the RF transceiver of a sensor tag may include one or more tag-printed coil antennas. In an example, the sensor (e.g., UV sensor) included in the sensor tag may enter an awake state when an amount of RF energy received at the RF transceiver (e.g., extracted RF energy) is greater than or equal to a threshold. In some aspects, in the awake state, the sensor tag (and the included sensor) may operate in an ultra-low power mode. For example, the sensor tag may implement ultra-low power sensor operations including dark current mitigation and cancellation for the sensor (e.g., UV sensor) included in the sensor tag, data conversion (e.g., analog-to-digital (ADC) conversion) of UV intensity measured at the sensor, data calibration, and data storage.

The UV disinfection system may support a global wake-up of sensor tags (and sensors thereof) included in the UV disinfection system. The global wake-up feature may enable the sensor tags to remain in low power mode or in hibernation mode to reduce power consumption. The sensor tags included in the UV disinfection system may be, for example, sensor tags located within a RF coverage area of the UV disinfection system (e.g., based on RF transmission power, RF signal quality, a distance threshold associated with the RF transceiver of the source device). In some aspects, the UV disinfection system may support temporal synchronization among the sensor tags included in the UV disinfection system. For example, via the RF transceiver, the source device may transmit temporal reference data (e.g., time data) to all sensor tags included in the UV disinfection system. In an example, each sensor tag may report UV levels measured by an included sensor, in combination with temporal information (e.g., timestamp data) corresponding to the measurements.

In some aspects, the source device may read digital data generated by the sensor tag. The sensor tag may communicate the digital data to the source device, for example, over an established communications link (e.g., RF communications link). In an example, the digital data may include UV radiation information and time information. For example, the digital data may include measured UV radiation levels and timestamps corresponding to when the UV radiation levels are measured by a sensor tag and/or read by the source device. In some examples, the sensor tags may be assigned respective unique identifiers, and the UV disinfection system may reference the identifiers to distinguish between UV data respectively measured by different sensor tags.

The UV disinfection system may include an image sensor. In an example, the image sensor and the radiation source may be positioned or located within the UV disinfection system such that, a relative position between the image sensor and the radiation source is configured or known. Based on the relative positioning, the image sensor and the radiation source may share a same frame of reference (e.g., a shared reference point). In some aspects, the image sensor may capture images of a physical environment to be sanitized by the UV disinfection system, using one or more captured images (e.g., background images of the physical environment) as a reference for determining characteristics associated with the physical environment, characteristics of sensor tags within the physical environment (e.g., distance from the image sensor and/or the radiation source to the sensor tags), or characteristics of physical objects within the physical environment (e.g., distance from the image sensor and/or the radiation source to the physical objects). In some examples, the image sensor may detect, calculate, and record geolocation information (e.g., coordinates, global positioning satellite (GPS) coordinates) of the sensor tags with respect to the physical environment. The geolocation information may include a local map or a local relative positioning of the sensor tags within a building.

In some aspects, each sensor tag may include a set of markers. The markers may also be referred to herein as UV markers, visual markers, luminous markers, targets, or points. In some cases, a set of markers may be referred to as an object (e.g., a set of four markers may be referred to as a four-point object). Each of the markers may include a UV coating (e.g., a UV luminous paint). In some cases, the image sensor may identify and locate a sensor tag based on the markers. In one embodiment, the marker may comprise a line and the four markers may include a non-symmetrical quadrilateral shape. For example, the image sensor may distinguish sensor tags from other reference objects in a physical environment, based on the markers included in the sensor tags. For example, the image sensor may identify the markers based on a response (e.g., of the respective UV coatings) to UV light emitted by the radiation source.

According to example aspects of the present disclosure, the UV disinfection system may emit UV radiation directed toward a target surface. In an example, the system may identify sensor tags and respective sensor tag locations through visual means (e.g., based on images captured by the image sensor and/or detection of respective markers). For example, for each sensor tag, the UV disinfection system may support visual calculation and estimation of location information (e.g., coordinates, positioning) of the respective markers. The UV disinfection system may determine direction and distance of the markers (and corresponding sensor tags), for example, in relation to the radiation source and/or the image sensor.

Based on the location information of the markers (and corresponding sensor tags), the UV disinfection system may apply RF beamforming techniques for directing RF signals from the source device to the sensor tags. In some cases, based on the location information, the UV disinfection system may apply the RF beamforming techniques to establish a communications link (e.g., a directed RF link) between the source device and the sensor tags. In some aspects, the directed RF link may support improved RF energy transfer (e.g., higher efficiency) to the sensor tags. In some other aspects, the directed RF link may support improved RF data communications (e.g., increased RF communications quality, increased quality of service (QoS)) between the source device and the sensor tags.

The UV disinfection system may collect and aggregate data from the sensor tags, in combination with identification information associated with the sensor tags. The identification information may include, for example, unique identifiers (UIDs) associated with the sensor tags. In some examples, the data may include measured UV radiation levels (also referred to herein as intensity information) and corresponding temporal information. Based on aggregating the data, the UV disinfection system may map topological information (e.g., location information, orientation information) associated with the sensor tags.

In some aspects, based on the aggregated data, the UV disinfection system may determine the amount of UV light incident a physical environment and/or any target surface of the physical environment. For example, the UV disinfection system may determine the amount of surface radiation (e.g., UV radiation coverage) and time duration (e.g., radiation duration) with respect to the physical environment and/or target surfaces. In some aspects, the system may process the aggregated data using any combination of data analytics, machine learning and artificial intelligence (AI) processing. In some examples, the UV disinfection system may transfer the aggregated data or any portion thereof to cloud-based data storage or a server (e.g., a cloud-based server) via wired or wireless communication.

A machine learning network (e.g., implemented in the UV disinfection system, the source device, or the server) may evaluate the data and generate feedback information (e.g., probability information, confidence information) with respect to the UV radiation coverage associated with the physical environment and/or target surface. For example, the machine learning network may generate and output feedback information with respect to the amount of UV radiation energy detected by radiation sensors of a sensor tag. In some aspects, the feedback information may be associated with the total amount of UV radiation energy detected by radiation sensors of multiple sensor tags (e.g., all sensor tags included in the physical environment). In some cases, based on an evaluation of the data, the machine learning network may predict the amount of UV radiation energy incident one or more target surfaces in the physical environment (e.g., UV radiation coverage) in relation to a predicted amount of time.

The system may collect and provide UV data to the machine learning network periodically (e.g., based on a schedule, a temporal duration, etc.) or in real-time. In some examples, based on the feedback information provided by the machine learning network, the system may set or adjust one or more parameters of the radiation source. For example, the system may adjust the output power of the radiation source, reposition the radiation source (e.g., modify an emission direction of the radiation source), or modify a location of the radiation source based on the feedback information.

In some aspects, the system may aggregate location information of the sensor tags. The system (or the machine learning network) may analyze the location information, for example, to verify the operation of the sensor tags. For example, the system may identify whether any sensor tags have been tampered with, incorrectly located by the system (e.g., not detected), or damaged. For example, the identification may be accomplished by comparing existing or previously stored marker data (e.g., location information associated with markers of the sensor tags) to current marker data. In yet another example, the geolocation information may be utilized for the same purpose (e.g., aggregating location information, analyzing location information). In some aspects, the system may identify whether any sensor tags are non-visible to the radiation source. For example, the system may identify whether an object located between the radiation source and a sensor tag is preventing UV light emitted by the radiation source from reaching the sensor tag.

The UV disinfection system may support synchronization between the source device and the sensor tags (e.g., time synchronization) in combination with various system level functionality. In some aspects, the UV disinfection system may support time synchronization between any of the source device, the RF transceiver of the source device, the radiation source, the image sensor, and the sensor tags in combination with system level functionality. For example, the UV disinfection system may support control (e.g., via commands, signals, etc.) of any of the source device, the RF transceiver of the source device, the radiation source, the image sensor, and the sensor tags. In an example, the UV disinfection system may support a central controller implemented at the source device or at a server (e.g., a cloud-based server, a local server) of the UV disinfection system.

In an example, the UV disinfection system may support controlling the emission of UV radiation based on the data aggregated from the sensor tags (e.g., as measured by the sensors). For example, the UV disinfection system may position the radiation source and/or direct emissions of the radiation source based on the data. In another example, the UV disinfection system may position and/or direct the RF transceiver of the source device based on the data aggregated from the sensor tags and image data captured by the image sensor (e.g., visual confirmation information by the image sensor with respect to sensor tag locations).

A sensor tag may include multiple markers. The system may detect the markers of the sensor tag (e.g., using the image sensor), and based on the detected markers, the system may identify location information of the sensor tag (or a radiation sensor included therein). For example, the system may use the location information to estimate the distance between the image sensor and the sensor tag. In another example, the system may calculate a viewing angle and/or distance from the image sensor to the sensor tag. Based on the relative positioning between the image sensor and the radiation source, the system may calculate an angle and/or distance from the radiation source to the sensor tag.

In some aspects, the markers may be UV luminous markers. For example, the markers may be formed of a radiation sensitive material disposed on a housing of the sensor tag. In an example, the radiation sensitive material may react (e.g., change color) in response to light having a wavelength in the UV-C wavelength range. For example, the radiation sensitive material may transition between different colors based on an exposure to UV-C light emitted by the radiation source.

In some examples, a sensor tag may include a minimum of four markers. In an example, the markers may represent four respective points (e.g., X-axis and Y-axis coordinates) in the Cartesian plane. Using the four points in a captured image, for example, the system (or machine learning network) may calculate or estimate the distance between the image sensor (or the radiation source) and the sensor tag. In one embodiment, the sensor tag may include four linear lines, and intersection points (e.g., representative of the four respective points) may be detected and recognized by the image sensor. In another aspect, using different images captured at different temporal instances, the machine learning network may predict a distance between the image sensor (or the radiation source) and the sensor tag with respect to a predicted temporal period (e.g., a future temporal instance). In some aspects, the system may support adjusting the resolution of the image sensor and/or optical zoom settings to increase the accuracy with respect to calculated distance and angle.

In some aspects, the markers on a sensor tag may be asymmetric with respect to a two-dimensional (2D) plane. For example, the markers may be asymmetric with respect to any axis associated with the sensor tag. Based on the asymmetry, for example, the image sensor may avoid capturing the same reference image (e.g., having the same marker positions and/or marker sizes) when capturing images of a sensor tag from different locations or perspectives. In an example of aspects of the present disclosure, the markers may be positioned on a sensor tag such that the markers are not located along shapes having an axis of symmetry (e.g., an ellipse).

In an example of a four-point object without an axis of symmetry, a set of four markers may be positioned asymmetric to one another. The four markers, if joined by imaginary lines, may form a polygon (e.g., a quadrilateral shape) in which the internal angles at each vertex are different from one another. In an example, with respect to the positioning of the four markers, one of the internal angles may be greater than 180 degrees.

According to other example aspects, the UV disinfection system described herein may be applied to personal protection equipment (PPE). For example, the sensor tags may be attachable to PPE.

Aspects of the subject matter described herein may be implemented to realize one or more advantages. For example, the described techniques may support improved UV radiation coverage and improved power savings compared to some UV disinfection systems.

Aspects of the disclosure are initially described in the context of a UV disinfection system. Examples of processes, system operations, and sensor configurations that support a UV disinfection system with sensors and feedback are then described. Aspects of the disclosure are further illustrated by and described with reference to apparatus diagrams, system diagrams, and flowcharts that relate to a UV disinfection system with sensors and feedback.

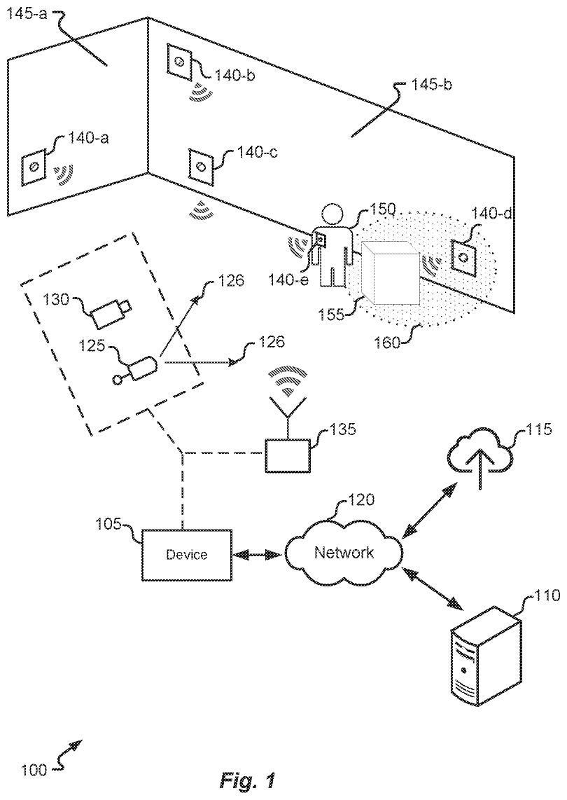

FIG. 1 illustrates an example of a system 100 that supports a UV disinfection system with sensors and feedback in accordance with aspects of the present disclosure. In some examples, the system 100 may be a UV disinfection system.

The system 100 may include a communication device 105 (or multiple communication devices 105), a server 110, a database 115, and a communication network 120. The communication device 105 may be referred to as a source wireless communication device. Non-limiting examples of the communication device 105 may include, for example, personal computing devices or mobile computing devices (e.g., laptop computers, mobile phones, smart phones, smart devices, wearable devices, tablets, etc.). In some examples, the communication device 105 may be operable by or carried by a human user. In some aspects, the communication device 105 may perform one or more operations autonomously or in combination with an input by the user, the communication device 105, and/or the server 110. In some aspects, the communication device 105 may be mounted on a transport instrument configured to patrol a target area.

The server 110 may be, for example, a cloud-based server. In some aspects, the server 110 may be a local server connected to the same network (e.g., Local Area Network (LAN), Wide Area Network (WAN), etc.) associated with the communication device 105. The database 115 may be, for example, a cloud-based database. In some aspects, the database 115 may be a local database connected to the same network (e.g., LAN, WAN) associated with the communication device 105 and/or the server 110. The database 115 may be supportive of data analytics, machine learning, and AI processing.

The communication network 120 may facilitate machine-to-machine communications between any of the communication device 105 (or multiple communication device 105), the server 110, or one or more databases (e.g., database 115). The communication network 120 may include any type of known communication medium or collection of communication media and may use any type of protocols to transport messages between endpoints. The communication network 120 may include wired communications technologies, wireless communications technologies, or any combination thereof.

The Internet is an example of the communication network 120 that constitutes an Internet Protocol (IP) network consisting of multiple computers, computing networks, and other communication devices located in multiple locations, and components in the communication network 120 (e.g., computers, computing networks, communication devices) may be connected through one or more telephone systems and other means. Other examples of the communication network 120 may include, without limitation, a standard Plain Old Telephone System (POTS), an Integrated Services Digital Network (ISDN), the Public Switched Telephone Network (PSTN), a LAN, a WAN, a wireless LAN (WLAN), a Session Initiation Protocol (SIP) network, a Voice over Internet Protocol (VoIP) network, a cellular network, and any other type of packet-switched or circuit-switched network known in the art. In some cases, the communication network 120 may include of any combination of networks or network types. In some aspects, the communication network 120 may include any combination of communication mediums such as coaxial cable, copper cable/wire, fiber-optic cable, or antennas for communicating data (e.g., transmitting/receiving data).

The system 100 may further include a radiation source 125, an image sensor 130, an RF transceiver 135 (also referred to herein as an RF transceiver device, an RF transponder device, or an RF transmitter-receiver device), and sensor tags 140 (e.g., sensor tag 140-a through sensor tag 140-d). In some aspects, the radiation source 125 and/or the image sensor 130 may be network capable devices capable of directly communicating with the communications network 120 (e.g., via a wired or wireless connection). For example, the radiation source 125 may be electrically coupled to the communication device 105 on a transport instrument configured to patrol a targeted area where the sensor tags 140 are located. In some other aspects, the radiation source 125 and/or the image sensor 130 may indirectly communicate with the communications network 120 (e.g., via the communication device 105).

The radiation source 125 may be a UV light source configured to emit light 126 (UV radiation) in the UV range. For example, the radiation source 125 may be configured to emit UV light associated with disinfecting the air and/or target surfaces 145 (e.g., target surface 145-a, target surface 145-b) of a physical environment to be sanitized by the system 100. The physical environment may include, for example, a hospital environment (e.g., a room in a hospital), a controlled environment (e.g., a clean room), a residential environment (e.g., a hotel room, a vacation rental), a commercial environment (e.g., a fitness facility, an office facility), or the like. In an example, the radiation source 125 may be configured to emit light having a wavelength between 100 nm to 280 nm (i.e., UV-C wavelength range).

The radiation source 125 may include a location sensor configured to record location information associated with the radiation source 125. In an example, the location sensor may be configured to record and output coordinates, positioning information, orientation information, velocity information, or the like. For example, the radiation source 125 may include an accelerometer, a GPS transponder, an RF transceiver, a gyroscopic sensor, or any combination thereof.

The image sensor 130 may be a single image sensor. In some aspects, the image sensor 130 may be an array of image sensors. The image sensor 130 may be integrated within the communication device 105 or a camera device. In an example, the image sensor 130 may be included in a standalone camera device or a camera device integrated with the communication device 105. The image sensor 130 may include photodiodes sensitive (e.g., capable of detecting) to light of a configured frequency band(s).

The camera device may be mechanically mounted to or within a housing of the communication device 105 in a manner that allows rotational degrees of freedom of the camera device and/or the image sensor 130. In another example, the camera device may be mounted to any surface or any object. In some aspects, the camera device may be a spherical camera device (e.g., for providing a spherical field of view).

The image sensor 130 may include a location sensor configured to record location information associated with the image sensor 130. In an example, the image sensor 130 may be configured to record and output coordinates, positioning information, orientation information, velocity information, or the like. For example, the image sensor 130 may include an accelerometer, a GPS transponder, an RF transceiver, a gyroscopic sensor, or any combination thereof.

The system 100 may include multiple cameras (and multiple image sensors 130). The multiple cameras (and multiple image sensors 130) may be integrated with the communication device 105 or multiple communication devices 105. In some aspects, the multiple cameras (and multiple image sensors 130) may be standalone cameras separate from the communication devices 105.

The image sensor 130 include any combination of photodiodes, photocathodes, and/or photomultipliers. The image sensor 130 may be configured to detect light within any defined wavelength range (e.g., visible spectrum, electromagnetic spectrum of ultraviolet radiation (UVR), etc.). In some aspects, the image sensor 130 may include one or more photodiodes implemented as UV sensors.

In an example, the image sensor 130 may be sensitive within the range of 200 nm to 1100 nanometers. In some examples, the image sensor 130 may include UV sensors that are sensitive to ultraviolet A (UV-A) radiation (e.g., having a wavelength from 315 nm to 400 nm), ultraviolet B (UV-B) radiation (e.g., having a wavelength from 280 nm to 315 nm), ultraviolet C (UV-C) radiation (e.g., having a wavelength from 100 nm to 280 nm), or any combination thereof.

The image sensor 130 and the radiation source 125 may be positioned or located within the system 100 such that, a relative position between the image sensor 130 and the radiation source 125 is configured or known (e.g., by the system 100, the communication device 105, the server 110, etc.). Based on the relative positioning, the image sensor 130 and the radiation source 125 may share a same frame of reference (e.g., a shared reference point).

In an example, any of the radiation source 125, the image sensor 130, and the RF transceiver 135 may be integrated within the communication device 105. For example, any of the radiation source 125, the image sensor 130, and the RF transceiver 135 may communicate within the communication device 105 over a system bus included in the communication device 105. In another example, any of the radiation source 125, the image sensor 130, and the RF transceiver 135 may be external to the communication device 105. For example, any of the radiation source 125, the image sensor 130, and the RF transceiver 135 may be electrically coupled (e.g., via a wired connection) to the communication device 105. In some examples, any of the radiation source 125, the image sensor 130, and the RF transceiver 135 may communicate with the communication device 105 via a wired connection, a wireless connection, or the communications network 120.

In some aspects, any of radiation source 125, the image sensor 130, and the RF transceiver 135 may be mechanically mounted to a transport instrument configured to move about the physical environment. In an example, movement of the transport instrument may be controlled by the system 100 (e.g., via commands by the communication device 105 or the server 110). In some other aspects, movement of the transport instrument may be autonomous or semi-autonomous (e.g., based on a schedule or programming). For example, the transport instrument may be instructed to patrol a target area associated with the physical environment. The transport instrument may be, for example, a mobile vehicle, a motorized robot, or the like.

The sensor tags 140 may be, for example, communication devices capable of transmitting and receiving signals (e.g., via wired or wireless communications). For example, the sensor tags 140 may communicate with the communication device 105 via an RF communications link established between the sensor tags 140 and the communication device 105. The sensor tags 140 may be referred to as sensor devices, receiver wireless communication devices, receiver devices, UV tags, or UV sensor tags.

Non-limiting examples of the sensor tags 140 may include, for example, Internet of Things (IoT) devices, wearable devices, or the like. For example, the sensor tags 140 may be disposed (e.g., attached, affixed, installed) on one or more target surfaces 145 (e.g., target surface 145-a, target surface 145-b) of the physical environment. In some other aspects, the sensor tags 140 may be operable by, carried by, or worn by a user 150. For example, a sensor tag 140 (e.g., sensor tag 140-e) may be attachable to PPE of a user 150. In some aspects, the sensor tags 140 may perform one or more operations autonomously or in combination with an input by the user, the communication device 105, the server 110, and/or a central controller.

Each of the sensor tags 140 may include one or more sensors. For example, each of the sensor tags 140 may include radiation sensors, light sensors, UV sensors, or electronic UV sensors. In an example, a sensor included in a sensor tag 140 may detect and measure UV radiation (e.g., UV-C radiation) emitted by the radiation source 125.

In some other examples, each of the sensor tags 140 may include a location sensor configured to record location information. In an example, the location sensor may be configured to record and output coordinates, positioning information, orientation information, velocity information, or the like. For example, the image sensor 130 may include an accelerometer, a GPS transponder, an RF transceiver, a gyroscopic sensor, or any combination thereof.

In some aspects, a sensor of a sensor tag 140 may convert detected UV radiation into digital data (e.g., a digital measurement value). In some examples, each sensor tag 140 may communicate measured UV radiation power at the sensor tag 140 (e.g., the total energy of UV radiation). Each sensor tag 140 may communicate the digital data (e.g., measured UV radiation power) to other devices in the system 100 using RF communications. For example, each sensor tag 140 may communicate the digital data to the communication device 105 (or other communication devices 105) via an RF transceiver included in the sensor tag 140. The sensor tag 140 may have an internal RF transceiver circuit configured to transmit data when the sensor tag 140 is within a visible range of the image sensor 130 (e.g., visible to the image sensor 130, within a threshold distance such that the sensor tag 140 is detectable by or visible to the image sensor 130). In this way, for example, data transmissions between the sensor tag 140 and the image sensor 130 can be performed with low energy (e.g., low transmission power) because the sensor tag 140 and the image sensor 130 (and the communication device 105) are in a close proximity (e.g., within a threshold distance of each other).

Based on digital data (e.g., digital measurement values) received or read from the sensor tags 140, the system 100 may calculate the total UV radiation (e.g., total energy) emitted by the radiation source 135 into the physical environment. In some aspects, the total UV radiation may include the total amount of UV radiation received at the sensor tags 140. In an example, based on the total amount of UV radiation received at the sensor tags 140, the system 100 may calculate or estimate the total UV radiation incident the physical environment (e.g., total UV radiation incident the target surfaces 145).

In some examples, a sensor tag 140 may be powered by an electrical power source electrically coupled to the sensor tag 140. For example, the electrical power source may include an external power source (e.g., an external power supply, a power outlet). In another example, the electrical power source may include batteries integrated with the sensor tag 140 (e.g., included in a housing of the sensor tag 140). The batteries may include long-life or extended-life batteries.

In some other aspects, the sensor tag 140 may be powered by energy harvesting techniques including solar, piezo-vibrational, or wireless charging. In an example, the sensor tag 140 may be powered by RF wireless waves (e.g., RF wireless charging). For example, the communication device 105 may wirelessly power or charge the sensor tag 140 using RF signals emitted from the RF transceiver 135. In some aspects, the system 100 may energize sensor tags 140 and sensors included therein (within an RF range of the RF transceiver 135), which may provide power to enable UV sensing operations of the sensor tags 140 and sensors. In some other aspects, the sensor tags 140 may be energized by background RF signals (e.g., Bluetooth, WiFi, 3G, 4G, or 5G sources) associated with the physical environment and/or RF signals within a threshold distance of the sensor tags 140.

Each sensor tag 140 may include multiple markers. The system 100 (e.g., communication device 105) may detect the markers of the sensor tag 140 using the image sensor 130, and based on the detected markers, the system 100 may identify location information of the sensor tag 140 (or a radiation sensor included in the sensor tag 140). For example, the system 100 may use the location information to estimate the distance between the image sensor 130 and the sensor tag 140. In another example, the system 100 may calculate a viewing angle and/or distance from the image sensor 130 to the sensor tag 140. Based on the relative known positioning between the image sensor 130 and the radiation source 125, the system 100 may calculate an angle and/or distance from the radiation source 125 to the sensor tag 140.

In some aspects, the markers may be UV luminous markers. For example, the markers may be formed of a radiation sensitive material disposed on a housing of the sensor tag 140. In an example, the radiation sensitive material may react (e.g., change color) in response to UV light emitted by the radiation source 125 (e.g., light having a wavelength in the UV-C wavelength range). For example, the radiation sensitive material may transition between different colors based on an exposure to the UV light emitted by the radiation source.

In some aspects, the image sensor 130 may capture images of the physical environment inclusive of the sensor tags 140 and/or target surfaces 145. The system 100 may use one or more captured images (e.g., captured images of the physical environment) as a reference for determining characteristics associated with the physical environment.

For example, the image sensor 130 may visually detect the sensor tags 140 based on the captured images. In some cases, the image sensor 130 may visually detect the sensor tags 140 based on radiation sensitive material (e.g., visual markers) disposed on the sensor tags 140.

Based on the captured images, the system 100 may determine characteristics of sensor tags 140 within the physical environment (e.g., distance from the image sensor 130 and/or the radiation source 125 to the sensor tags). For example, the system 100 may determine location information, orientation information, velocity information (e.g., for examples in which a sensor tag 140 is attached to a moving object or a user), and/or identification information associated with the sensor tags 140. In some examples, the system 100 may determine characteristics of physical objects within the physical environment (e.g., distance from the image sensor 130 and/or the radiation source 125 to the physical objects).

In some other examples, the system 100 (e.g., using the image sensor 130) may detect and record geolocation information (e.g., coordinates, GPS) coordinates) of the sensor tags 140 with respect to the physical environment. For example, the system 100 may detect and record the geolocation information based on location information of the image sensor 130 and the calculated angle and distance from the image sensor 130 to the sensor tag 140. In some aspects, the system 100 may detect and record the geolocation information based on location information of the communication device 105 (when the image sensor 130 is integrated with the communication device 105).

The communication device 105 may communicate (e.g., transmit or receive) data packets with one or more other devices of the system 100. For example, the communication device 105 may exchange data packets with another communication device 105, the server 110, the database 115, the radiation source 125, the image sensor 130, or the RF transceiver 135, or the via the communication network 120. In some examples, the communication device 105 may communicate with another device (e.g., another communication device 105, database 115, radiation source 125, image sensor 130, RF transceiver 135) via the server 110.

The system 100 may support a global wake-up of the sensor tags 140 (e.g., wake-up of sensors included in the sensor tags 140). The sensor tags 140 (and sensors) may be, for example, located within a RF coverage area of the system 100. In some aspects, the RF coverage area may be based on RF transmission power of the RF transceiver 135, RF signal quality of a communications link (e.g., an RF communications link) established between the RF transceiver 135 and the sensor tags 140 and/or a distance threshold from the RF transceiver 135.

In some aspects, the system 100 may support temporal synchronization among the sensor tags 140. For example, the communication device 105 (e.g., via the RF transceiver 135) may transmit temporal reference data (e.g., time data) to all the sensor tags 140 (e.g., periodically, based on a schedule, on demand, as part of a global wake-up event, etc.). The sensor tags 140 may synchronize with the temporal reference data. In an example, each sensor tag 140 (e.g., sensor tag 140-a through sensor tag 140-d) may report UV levels measured by an included sensor, in combination with temporal information (e.g., timestamp data) corresponding to the measurements. The timestamp data may be synchronized with the temporal reference data.

In some aspects, using the RF transceiver 135, the communication device 105 may read digital data generated by each sensor tag 140. The sensor tags 140 may communicate respective digital data to the communication device 105, for example, over an established communications link or via broadcast communications. In an example, the digital data may include UV radiation information and time information. For example, the digital data may include measured UV radiation levels and timestamps corresponding to when the UV radiation levels are measured by a sensor tag 140 and/or read by the communication device 105. In some examples, the sensor tags 140 may be assigned respective unique identifiers, and the UV disinfection system may reference the identifiers to distinguish between UV data respectively measured by each sensor tag 410.

According to example aspects of the present disclosure, the system 100 may emit UV radiation directed toward a target surface 145. In an example, the system 100 may identify sensor tags 140 and respective sensor tag locations through visual means (e.g., based on images captured by the image sensor 130 and/or detection of respective markers). For example, for each sensor tag 140, the system 100 may support visual calculation and estimation of location information (e.g., coordinates, positioning) of the respective markers. The system 100 may determine direction and distance of the markers (and corresponding sensor tags 140), for example, in relation to the radiation source 125 and/or the image sensor 130.

Based on the location information of the markers (and corresponding sensor tags 140), the system 100 may apply RF beamforming techniques for directing RF signals from the communication device 105 to the sensor tags 140. In some cases, based on the location information, the system 100 may apply the RF beamforming techniques to establish a communications link (e.g., a directed RF link) between the communication device 105 and the sensor tags 140. In some aspects, the directed RF link may support improved RF energy transfer (e.g., higher efficiency) to the sensor tags 140. In some other aspects, the directed RF link may support improved RF data communications (e.g., increased RF communications quality, increased QoS) between the communication device 105 and the sensor tags 140.

The system 100 may collect and aggregate data from the sensor tags 140, in combination with identification information associated with the sensor tags 140. The identification information may include, for example, unique identifiers (UIDs) associated with the sensor tags 140. In some examples, the data may include measured UV radiation levels and corresponding temporal information. Based on aggregating the data, the system 100 may map topological information (e.g., location information, orientation information) associated with the sensor tags 140.

In some aspects, based on the aggregated data, the system 100 may determine the amount of UV light incident a physical environment and/or any target surface 145 of the physical environment. For example, the system 100 may determine the amount of surface radiation (e.g., UV radiation coverage) and time duration (e.g., radiation duration) with respect to the physical environment and/or target surfaces 145. In some aspects, the system 100 may process the aggregated data using any combination of data analytics, machine learning and AI processing. In some examples, the system 100 may transfer the aggregated data or any portion thereof to cloud-based data storage or the server 110 via wired or wireless communication.

A machine learning network (e.g., implemented in the system 100, the communication device 105, or the server 110) may evaluate the data and generate feedback information (e.g., probability information, confidence information) with respect to the UV radiation coverage with respect to the physical environment and/or target surface. For example, the machine learning network may generate and output feedback information with respect to the amount of UV radiation energy detected by radiation sensors of a sensor tag 140. In some aspects, the feedback information may be associated with the total amount of UV radiation energy detected by radiation sensors of multiple sensor tags 140 (e.g., all sensor tags 140 included in the physical environment). In some cases, based on an evaluation of the data, the machine learning network may predict the amount of UV radiation energy incident one or more target surfaces in the physical environment.

In an example, based on the aggregated data and/or images captured by the image sensor 130, the machine learning network may output predicted radiation coverage corresponding to a target area of the physical environment and a temporal period. In some examples, the output may include probability information corresponding to the predicted radiation coverage and/or confidence information associated with the probability information. In some aspects, the system 100 (e.g., using the machine learning network) may calculate or suggest relatively shorter temporal periods for applying UV radiation compared to some other UV disinfection systems.

In another example, based on the aggregated data and/or images captured by the image sensor 130, the machine learning network may output predicted location information and/or predicted movement associated with the sensor tags 140 and a predicted temporal period. In some aspects, the output may include predicted orientation information associated with the sensor tags 140 and the predicted temporal period. In some other aspects, the output may include predicted velocity information associated with the sensor tags 140 and the predicted temporal period. The output may include probability information and/or confidence information associated with the predicted location information, the predicted orientation information, the predicted velocity information, and/or the predicted temporal period.

Based on the output from the machine learning network, the system 100 (e.g., central controller) may control a location of the radiation source 125, an emission direction of the radiation source 125, an emission power of the radiation source 125, and/or an emission duration of the radiation source 125.

The system 100 may collect and provide UV data to the machine learning network periodically (e.g., based on a schedule, a temporal duration, etc.) or in real-time. In some examples, based on the feedback information provided by the machine learning network, the system 100 may set or adjust one or more parameters of the radiation source 125. For example, the system 100 may adjust the output power of the radiation source 125, reposition the radiation source 125 (e.g., modify an emission direction of the radiation source 125), or modify a location of the radiation source 125 based on the feedback information.

In some aspects, the system 100 may aggregate location information of the sensor tags 140. The system 100 (or the machine learning network) may analyze the location information, for example, to verify the operation of the sensor tags 140. For example, the system 100 may identify whether any sensor tags 140 have been tampered with, incorrectly located by the system 100 (e.g., not detected), or damaged. In some aspects, the system 100 may identify whether any sensor tags 140 are non-visible to the radiation source 125. For example, the system 100 may identify whether an object located between the radiation source 125 and a sensor tag is preventing UV light emitted by the radiation source 125 from reaching the sensor tag 140.

The system 100 may support synchronization between the communication device 105 and the sensor tags 140 (e.g., time synchronization) in combination with various system level functionality. In some aspects, the system 100 may support synchronization between any of the communication device 105, the RF transceiver 135 of the communication device 105, the radiation source 125, the image sensor 130, and the sensor tags 140 in combination with system level functionality. For example, the system 100 may support control (e.g., via commands, signals, etc.) of any of the communication device 105, the RF transceiver 135 of the communication device 105, the radiation source 125, the image sensor 130, and the sensor tags 140.

In an example, the system 100 may support a central controller (e.g., a central processing device). In some aspects, the central controller may be implemented at the communication device 105 or the server 110 of the system 100. For example, the central controller may be included in a processor of the communication device 105 or the server 110. In an example, the central controller may control multiple devices (e.g., communication devices 105, servers 110, radiation source 125, image sensor 130, sensor tags 140, etc.) included in the system 100, where each of the devices is associated with a respective function or task. In some cases, each of the devices may transmit status information and/or data to the central controller. In some aspects, each of the devices may receive actuating signals from the central controller in association with performing a respective function or task.

In an example in which the central controller is implemented at the server 110, the central controller may communicate with another communication device 105, another server 110, the radiation source 125, the image sensor 130, or the sensor tags 140 via the communication device 105 and/or the communications network 120. Example aspects of the system 100 described herein may be performed by the central controller in combination with devices of the system 100.

In an example, the system 100 (e.g., via the central controller) may support aggregation of the intensity information recorded by the sensor tags 140. In some examples, the system 100 may maintain a record of the intensity information recorded by the sensor tags 140. The system 100 may use the intensity information to compute information indicative of whether the target area is sufficiently disinfected (e.g., the UV radiation at the target area satisfies a disinfection level or a disinfection coverage, a pathogen level at the target area satisfies a threshold, etc.). In some aspects, the sensor tags 140 may transmit the intensity information to the central controller wirelessly (e.g., via an RF communications link). The intensity information may form a portion of a safety control sub-system of the system 100. For example, when a human subject is present within the target area, the system 100 may be configured to output a notification (e.g., sound an alarm) to warn the human subject about the safety level of the radiation (e.g., based on a detected intensity information exceeding a threshold).

In some aspects, the system 100 may evaluate one or more parameters associated with the radiation source 125 based on the aggregated intensity information. In some aspects, the one or more parameters may include a degradation level associated with the UV radiation emitted by the radiation source 125, a degradation rate associated with the emitted UV radiation, or both. In some aspects, the radiation source 125 may degrade over time (e.g., based on usage, manufacturing age, etc.), and when the degradation exceeds a predetermined level, the radiation source 125 may have to be replaced for being unable to sufficiently disinfect the entire target area. In some other aspects, the one or more parameters associated may include a quality level of a disinfection session implemented by the system 100. In some cases, the sensor tags 140 output a notification associated with the quality level of the disinfection session compared to a quality level threshold.

In some aspects, the system 100 (e.g., via the central controller) may support controlling the emission of UV radiation based on the data aggregated from the sensor tags 140 (e.g., as measured by the sensors). For example, the system 100 may position the radiation source 125 and/or direct emissions of the radiation source 125 based on the data. In another example, the system 100 may position and/or direct the RF transceiver 135 of the communication device 105 based on the data aggregated from the sensor tags 140 (e.g., aggregated intensity information) and image data captured by the image sensor 130 (e.g., visual confirmation information with respect to sensor tag locations). Accordingly, for example, the system 100 may control a location of the radiation source 125, an emission direction of the radiation source 125, an emission power of the radiation source 125, or a combination thereof.

In some aspects, the sensor tags 140 may record temporal information (e.g., timestamp values) associated with the intensity information measured by sensors included in the sensor tags 140. In an example, the system 100 may evaluate the one or more parameters associated with the radiation source 125 based on the temporal information.

In some examples, the system 100 may support aggregation of intensity information from multiple sensor tags 140 based on temporal information (e.g., timestamp values) associated with the intensity information. For example, the sensor tag 140-a may record first intensity information associated with UV radiation received at the sensor tag 140-a, along with a timestamp value associated with the first intensity information. The sensor tag 140-b may record second intensity information associated with UV radiation received at the sensor tag 140-b, along with a timestamp value associated with the second intensity information. The system 100 may aggregate the first intensity information and the second intensity information based on comparison of the timestamp values. For example, the system 100 may aggregate the first intensity information and the second intensity information based on whether a difference between the respective timestamp values is below a temporal threshold.

According to other example aspects of the present disclosure, the central controller may be configured to receive interrupt signals from the sensor tags 140. For example, each sensor tag 140 may generate an interrupt signal when UV radiation measured at the sensor tag 140 (e.g., radiation intensity level measured at a radiation sensor of the sensor tag 140) is above a predetermined overlimit level.

In an example, the sensor tag 140-e (attached to PPE of the user 150) may generate and transmit an interrupt signal (e.g., a pause command, an indicator) to the central controller when UV radiation measured at the sensor tag 140-e is above a predetermined safety level associated with human exposure to the UV radiation. In response to the interrupt signal, the central controller may pause UV radiation transmissions (e.g., for a temporal period, intermittently, etc.) by the radiation source 125 and/or reduce emission power until the UV radiation measured at the sensor tag 140-e is below the predetermined safety level. In an example, the sensor tag 140-e may transmit a signal (e.g., a resume command, an indicator) to the central controller to continue UV radiation transmissions by the radiation source 125 and/or increase emission power (e.g., to a configured power level, a previous power level, etc.).

In some aspects, the sensor tag 140-e may directly alert the user 150 that the measured UV radiation is above the predetermined safety level. For example, the sensor tag 140-e may generate and output any combination of audible (e.g., via a speaker), visual (e.g., via a display, a light emitting diode (LED), or a color transition of a marker disposed on the sensor tag 140-e), or haptic notifications for alerting the user. In another example, the sensor tag 140-e may indirectly alert the user 150 via the central controller. For example, the sensor tag 140-e may transmit the interrupt signal to the central controller, and the central controller may control the communication device 105 to alert the user 150.

For example, the communication device 105 may be a mobile device worn or carried by the user 150, and the communication device 105 may output an audible, visual, and/or haptic notification to the user 150. In some other aspects, the sensor tag 140-e may passively alert the user 150 via a radiation sensitive material disposed on the sensor tag 140-e.

In another example, a sensor tag 140 may generate and transmit a signal (e.g., a pause command, a resume command, an indicator) based on a pathogen level detected by the sensor tag 140 with respect to a threshold. In response to the signal, the central controller may interrupt UV radiation transmissions (e.g., based on a pause command), resume UV radiation transmissions (e.g., based on a resume command), increase emission power of the radiation source 125 (e.g., based on an indication that pathogen levels are above a threshold), and/or increase the duration for emitting UV radiation from the radiation source 125 (e.g., based on an indication that pathogen levels are above a threshold).

In some aspects, the central controller may evaluate the radiation source 125 using measured UV radiation levels (also referred to herein as measured UV intensity or intensity information) provided by sensor tags 140 that are visible to the image sensor 130. In some examples, the central controller may position or relocate the image sensor 130 (e.g., using a transport instrument coupled to the image sensor 130) for cases in which a physical object between the image sensor 130 and a sensor tag 140 prevents the image sensor 130 from visually detecting the sensor tag 140.

For example, an object 155 may be located between the image sensor 130 and the sensor tag 140-d such that the sensor tag 140-d is not visible to the image sensor 130. Based on previously recorded UV radiation measurements and/or images previously captured by the image sensor 130, the central controller may be aware of the presence of the sensor tag 140-d. However, based on a current image (e.g., a static image, a video image) captured by the image sensor 130, the central controller may identify that the sensor tag 140-d is absent from a visual zone 160 associated with the captured image. The central controller may position or relocate the image sensor 130 (e.g., using the transport instrument coupled to the image sensor 130) until the image sensor 130 visually detects the sensor tag 140-d. Once the sensor tag 140-d is visually detected, the central controller may evaluate the radiation source 125 using the UV radiation measurements from the sensor tag 140-d.

In another example, the object 155 may be located between the RF transceiver 135 and the sensor tag 140-d such that RF communications between the RF transceiver 135 and the sensor tag 140-d are obstructed. Based on previously recorded UV radiation measurements received by the RF transceiver 135 and/or images previously captured by the image sensor 130, the central controller may be aware of the presence of the sensor tag 140-d. However, based on a current reading via the RF transceiver 135, the central controller may identify that RF communications between the RF transceiver 135 and the sensor tag 140-d are obstructed. The central controller may position or relocate the RF transceiver 135 (e.g., using the transport instrument coupled to the RF transceiver 135) until an RF communications link is reestablished with the sensor tag 140-d.

In some other aspects, the central controller may compute a distance between the radiation source 125 (or image sensor 130) and a sensor tag 140 using the example techniques described herein. The central controller may compare the distance to the UV radiation levels measured at the sensor tag 140 so as to compute an evaluation result regarding the radiation source 125 (e.g., UV radiation intensity, UV radiation power, UV radiation coverage, UV radiation transmission efficiency, etc.). In some examples, the evaluation result may include degradation information of the radiation source 125. In some other examples, the evaluation result may include an evaluation result of whether an area adjacent to a sensor tag 140 is sufficiently disinfected (e.g., the amount of pathogens present on a target surface 145 on which a sensor tag 140 is disposed is below a threshold).

In some cases, the central controller may position and/or relocate the radiation source 125 (e.g., using a transport instrument coupled to the radiation source 125) based on a predetermined threshold corresponding to a UV radiation intensity for removing pathogens from the physical environment (e.g., a target area, a target surface 145-a). The predetermined threshold may be referred to as a germ-kill limit. In an example, the central controller may position and/or relocate the radiation source 125 such that a distance between the sensor tag 140-a and the radiation source 125 is reduced. The central controller may adjust the location, an emission direction, and/or an emission power of the radiation source 125, for example, until the measured UV radiation at the sensor tag 140-a (and thereby the UV radiation at the target surface 145-a) exceeds the predetermined threshold. In some cases, the central controller may pause (or unpause) UV emissions and/or reduce (or increase) emission power of the radiation source 125 based on a comparison of the measured UV radiation at the sensor tag 140-a (and thereby the UV radiation at the target surface 145-a) to the predetermined threshold.

In some aspects, the central controller may maintain location records of the sensor tags 140 in the physical environment. The central controller may generate and/or update the location records based on images captured by the image sensor 130 and/or UV radiation measurements reported to the central controller by the sensor tags 140. In some aspects, based on the captured images and/or UV radiation measurements (or the location records generated therefrom), the central controller may identify whether any of the sensor tags 140 are inactive, damaged, not present in the physical environment, etc. The central controller may alert the user 150 (or any components of the system 100) of any sensor tags 140 that the central controller identifies as inactive, damaged, or not present in the physical environment.

While the illustrative aspects, embodiments, and/or configurations illustrated herein show the various components of the system 100 collocated, certain components of the system 100 can be located remotely, at distant portions of a distributed network, such as a LAN and/or the Internet, or within a dedicated system. Thus, it should be appreciated, that the components of the system 100 can be combined in to one or more devices or collocated on a particular node of a distributed network, such as an analog and/or digital telecommunications network, a packet-switched network, or a circuit-switched network. It will be appreciated from the following description, and for reasons of computational efficiency, that the components of the system 100 can be arranged at any location within a distributed network of components without affecting the operation of the system 100.

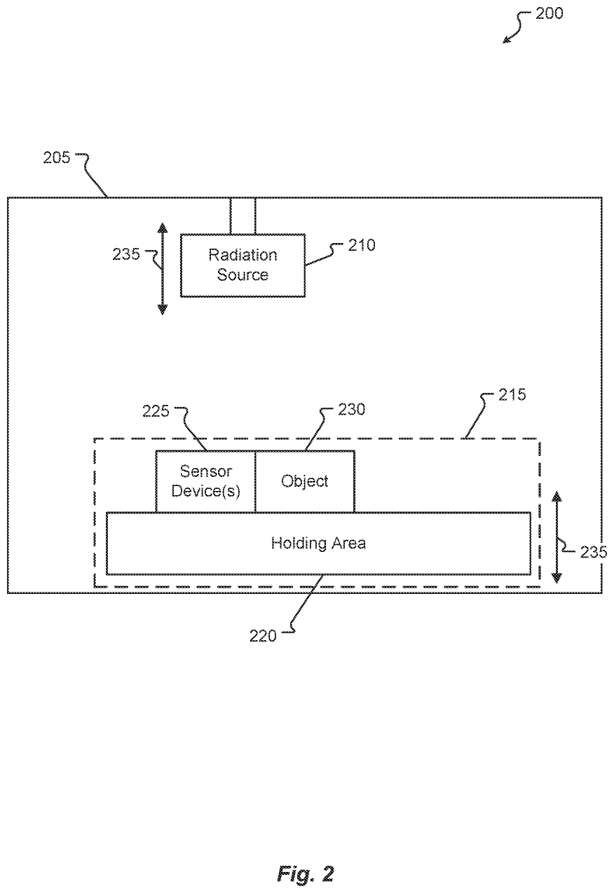

FIG. 2 illustrates an example of a system 200 that supports a UV disinfection system with sensors and feedback in accordance with aspects of the present disclosure. In some examples, the system 200 may include an enclosure 205; a radiation source 210 configured to emit UV radiation located within the enclosure 205 towards a targeted area 215; a holding area 220 located within the targeted area 215 configured to receive the UV radiation; and one or more sensor devices 225, the one or more sensor devices 225 including at least a first sensor configured to detect the UV radiation having a UV profile. Additionally, the system 200 may include an object 230 that is to be radiated. In some aspects, the one or more sensor devices 225 may be configured to record intensity information associated with the UV radiation. Additionally, one of the radiation source 210 and the holding area 220 may be fixed within the enclosure 205, and the other one of the radiation source 210 and the holding area 220 may be movable (e.g., along a direction 235) so as to adjust an intensity of the UV radiation received at the one or more sensor devices 225 (e.g., and at the object 230) by way of controlling a distance between the holding area 220 and the radiation source 210.

In some examples, the one or more sensor devices 225 of the system 200 may further include an additional sensor (e.g., at least a second sensor) configured to detect the UV radiation having an additional UV profile. For example, the UV profile corresponding to the UV radiation detected by the first sensor may be wider than the additional UV profile corresponding to the UV radiation detected by the additional sensor.

In some examples, the enclosure 205 may include a compartment of a building or a vehicle.

In some examples, the radiation source 210 may be attached adjacent to a light source located within the enclosure 205. Additionally, the radiation source 210 may be attached to a movable structure configured to be lower down from a top surface of the enclosure 210. For example, the radiation source 210 may be attached to the movable structure from the ceiling of the enclosure 210, such that the radiation source moves along the direction 235. Additionally or alternatively, the radiation source 210 may be attached to a movable robot configured to approach the targeted area 215. In some examples, the movable robot may be further configured to approach one or more additional targeted areas of one or more additional enclosures which are positioned at fixed location(s) relative to the enclosure 205 and the one or more additional enclosures.

In some examples, while not shown, the system 200 may further include: one or more additional radiation sources configured to emit the UV radiation; one or more additional sensor devices positioned opposing the one or more additional radiation sources within the targeted area, the one or more additional sensor devices configured to record the intensity information associated with the UV radiation; and a controller configured to communicate with each of the additional radiation sources and the one or more additional sensor devices. In some examples, the controller may be configured to control and maintain an aggregate value of the intensity information for each of the one or more sensor devices and the one or more additional sensor devices to be above a predetermined value.

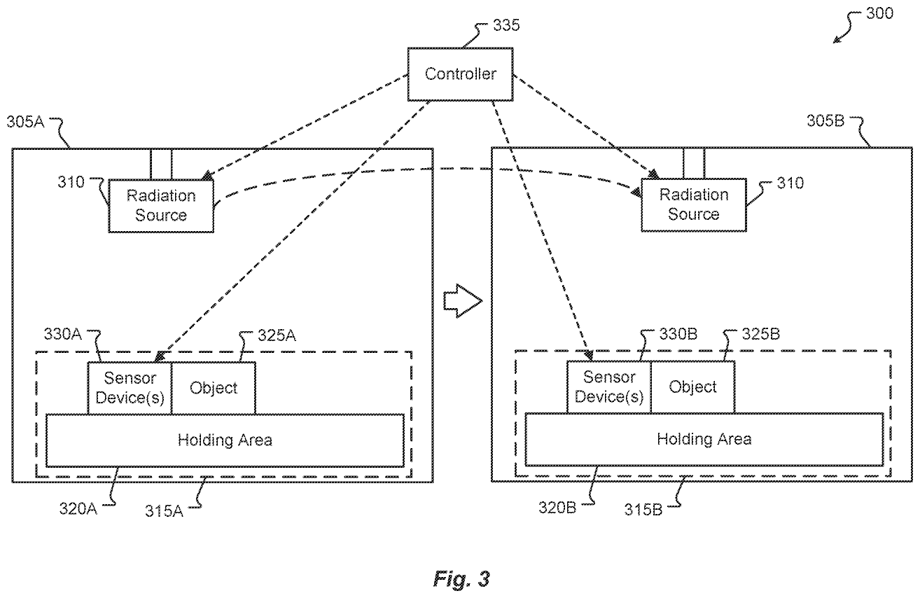

FIG. 3 illustrates an additional example of a system 300 that supports a UV disinfection system with sensors and feedback in accordance with aspects of the present disclosure. In some examples, the system 300 may include a plurality of enclosures 305 (e.g., at least a first enclosure 305A and a second enclosure 305B). Each of the plurality of enclosures 305 may include: a radiation source 310 configured to emit UV radiation towards targeted areas 315; holding areas 320 located within the targeted areas 315, where the holding areas 320 are configured to receive the UV radiation (e.g., such that objects 325 placed within the holding areas 320 receive the UV radiation); one or more sensor devices 330 (e.g., including at least a first sensor configured to detect the UV radiation and configured to record intensity information associated with the UV radiation); and a controller 335 configured to communicate with each of the radiation source 310 and the one or more sensor devices 330 of the plurality of enclosures 305. In some examples, the controller 335 may be configured to control and maintain an aggregate value of each of the intensity information recorded from each of the one or more sensor devices 330 above a predetermined value.

In some examples, the controller 335 is configured to maintain the aggregate value above the predetermined value by adjusting a time period of the targeted areas 315 being exposed to the UV radiation of each of the plurality of enclosures 305. In some examples, each of the plurality of enclosures 305 may include individual radiation sources in each enclosure 305. Additionally or alternatively, the system 300 may include a single radiation source that can be moved between each of the plurality of enclosures 305. For example, the single radiation source may be attached to a movable robot configured to approach the targeted area and an additional targeted area of an additional enclosure which are positioned at a fixed location relative to the enclosure and the additional enclosure.

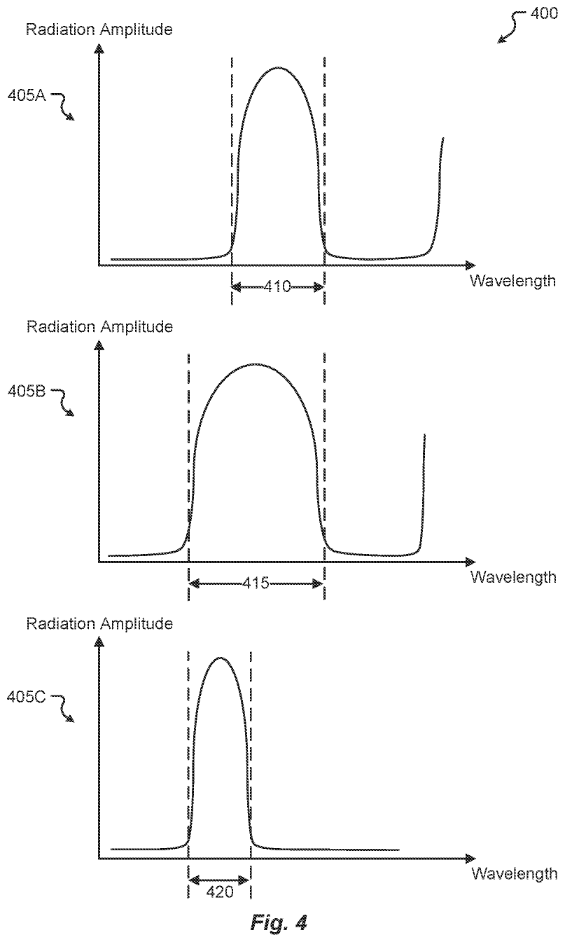

FIG. 4 illustrates example filter profiles 400 that support a UV disinfection system with sensors and feedback in accordance with aspects of the present disclosure. For example, the example filter profiles 400 may include a first filter profile 405A, a second filter profile 405B, and a third profile 405C.

In some examples, as described previously with reference to FIG. 2, a system may include an enclosure, a radiation source configured to emit UV radiation within the enclosure towards a targeted area, a holding area located within the targeted area configured to receive the UV radiation (e.g., including an object to be radiated), and one or more sensor devices. In some examples, the one or more sensor devices may include a first sensor configured to detect the UV radiation having the first filter profile 405A and a second sensor configured to detect the UV radiation having the second filter profile 405C. In some examples, the second filter profile 405B may allow a wider band 415 of UV radiation to pass through as compared to a band 410 of the first filter profile 405A. In some examples, the one or more sensor devices may be configured to record intensity information associated with the UV radiation.

In some examples, the one or more sensor devices may further include an arithmetic unit configured to perform either a subtraction or an additional function to outputs of the first sensor and the second sensor. For example, the arithmetic unit may perform the subtraction or additional function to generate the third filter profile 405C that includes a band 420 (e.g., the band 420 may correspond to the subtraction or additional function being performed using the bands 410 and 415).

In some examples, the one or more sensor devices may further include a first UV filter associated with the first filter profile 405A that is configured to pass through radiation with wavelengths between 300 nm and 400 nm. Additionally, the one or more sensor devices may further include a second UV filter associated with the second filter profile 405B that is configured to pass through radiation with wavelengths between 220 nm and 400 nm. In some examples, the first UV filter and the second UV filter may have an overlapping wavelength band within a UV wavelength range of 200 nm and 400 nm. Additionally or alternatively, the first filter profile 405A and the second filter profile 405B have an overlapping wavelength band within a UV wavelength range of 300 nm and 400 nm.

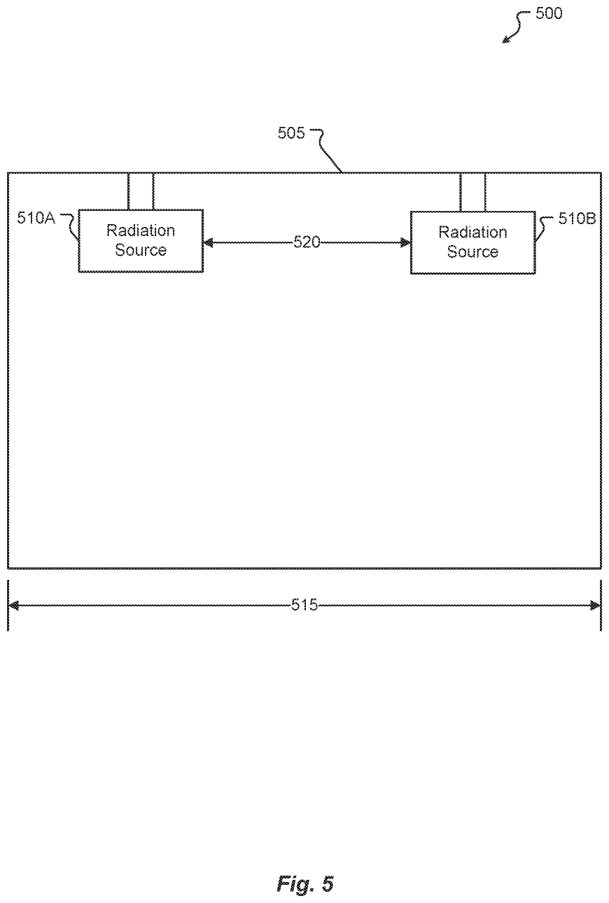

FIG. 5 illustrates a system configuration 500 that supports a UV disinfection system with sensors and feedback in accordance with aspects of the present disclosure.

In some examples, as described and previously shown with reference to FIGS. 2 and 3, a UV disinfection system may include: an enclosure 505; one or more radiation sources 510 configured to emit UV radiation; one or more sensor devices positioned opposing the one or more radiation sources 510 within a targeted area, the one or more sensor devices configured to record intensity information associated with the UV radiation; and a controller configured to communicate with each of the radiation sources 510 and the one or more sensor devices. For example, the system configuration 500 may include at least a first radiation source 505A and a second radiation source 505B positioned in the enclosure 505.

In some examples, the enclosure 505 may have a diameter 515, and the one or more radiation sources 510 may be positioned such that a distance 520 between any adjacent two of the one or more radiation sources 510 is at least 60% of a length of the diameter 515.

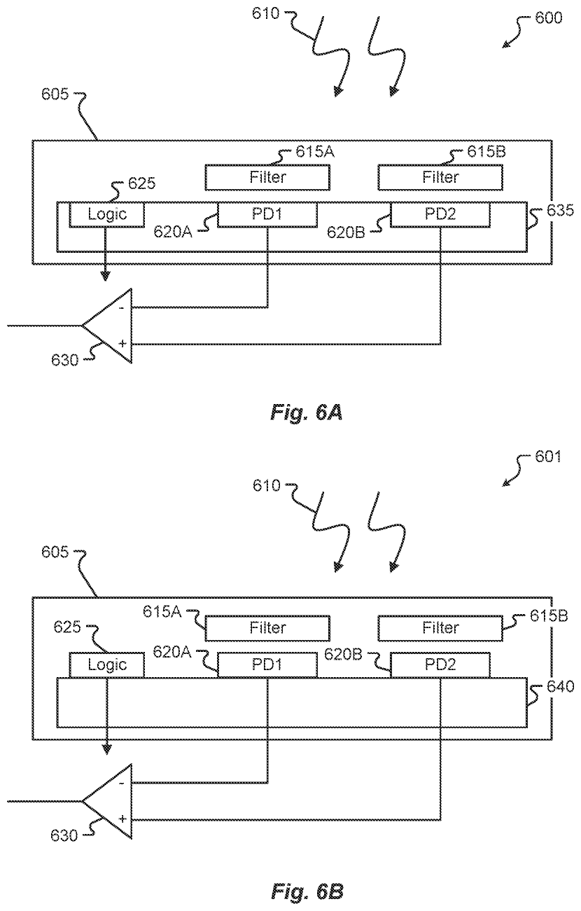

FIGS. 6A and 6B illustrate example housing configurations 600 and 601, respectively, that support a UV disinfection system with sensors and feedback in accordance with aspects of the present disclosure.

In some examples, a sensing device is provided that includes: a housing 605 configured to receive an external radiation 610 from a first direction; a first sensor 620A (e.g., such as a first photodetector (PD1)) configured to detect an UV radiation having a first filter profile; a first UV filter 615A positioned adjacent to the first sensor 620A within the housing 605 such that a first radiation from the first direction received by the first sensor 620A is configured to pass through the first UV filter 615A; a second sensor 620B (e.g., such as a second photodetector (PD2)) configured to detect the UV radiation having a second filter profile, the second filter profile allowing a wider band of UV radiation to pass through as compared to the first filter profile; a second UV filter 615B positioned adjacent to the second sensor 620B within the housing 605 such that a second radiation from the first direction received by the second sensor 620B is configured to pass through the second UV filter; and a controller 625 (e.g., providing logic for the sensing device). In some examples, the controller 625 is configured to generate and output in accordance with the first radiation received at the first sensor 620A and the second radiation received at the second sensor 620B.

In some examples, the sensing device may also include an arithmetic unit 635 configured to perform either a subtraction or an additional function to outputs of the first sensor 620A and the second sensor 620B. In some examples, the sensing device may further include a control block configured to provide an indication that a wavelength of the UV radiation is inside an overlapping wavelength band. Additionally or alternatively, the sensing device may further include a control block configured to provide an indication that a wavelength of the UV radiation is outside the overlapping wavelength band. In some examples, the first UV filter 615A and the second UV filter 615B may allow radiation of infra-red bandwidths to pass through.

In some examples, as shown in the example of the housing configuration 600 of FIG. 6A, the housing 605 may include an opto-semiconductor package 630. In these examples, the first sensor 620A includes a first die, the second sensor 620B includes a second die, and the controller 625 includes a third die. Additionally, the first die, the second die, and the third die may be located within the opto-semiconductor package 630.

Additionally or alternatively, as shown in the example of the housing configuration 601 of FIG. 6B, the sensing device may include a semiconductor chip 640. In some examples, the first sensor 620A may include a first photo-sensing area of the semiconductor chip 640, and the second sensor 620B may include a second photo-sensing area of the semiconductor chip 640. Additionally, the controller 625 may include a logic circuit area of the semiconductor chip 640.

Any of the steps, functions, and operations discussed herein can be performed continuously and automatically.

The exemplary systems and methods of this disclosure have been described in relation to examples of a system 100 (e.g., communication device 105, radiation source 125, image sensor 130, RF transceiver 135, sensor tags 140). However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

Furthermore, while the exemplary embodiments illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed embodiments, configuration, and aspects.

A number of variations and modifications of the disclosure can be used. It would be possible to provide for some features of the disclosure without providing others.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the embodiments with reference to particular standards and protocols, the disclosure is not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an embodiment that is entirely hardware, an embodiment that is entirely software (including firmware, resident software, micro-code, etc.) or an embodiment combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A system, comprising:
an enclosure;
a radiation source configured to emit ultraviolet (UV) radiation located within the enclosure towards a targeted area;
a holding area located within the targeted area configured to receive the UV radiation; and
one or more sensor devices, the one or more sensor devices comprising a sensor configured to detect the UV radiation having a UV profile, wherein the one or more sensor devices are configured to record intensity information associated with the UV radiation;
wherein one of the radiation source and the holding area is fixed within the enclosure and the other one of the radiation source and the holding area is movable so as to adjust an intensity of the UV radiation received at the one or more sensor devices by way of controlling a distance between the holding area and the radiation source.

2. The system of claim 1, wherein the one or more sensor devices further comprise an additional sensor configured to detect the UV radiation having an additional UV profile, wherein the UV profile is wider than the additional UV profile.

3. The system of claim 2 wherein the UV profile allows a first radiation with wavelengths between 220 nanometers (nm) and 400 nm to substantially passthrough, and wherein the additional UV profile allows a second radiation with wavelengths between 300 nm and 400 nm to substantially pass through.

4. The system of claim 1, wherein the enclosure includes a compartment of a building or a vehicle, and wherein the radiation source is attached adjacent to a light source located within the enclosure, and wherein the radiation source is attached to a movable structure configured to be lowered down from a top surface of the enclosure.

5. The system of claim 1, wherein the radiation source is attached to a movable robot configured to approach the targeted area and an additional targeted area of an additional enclosure which are positioned at a fixed location relative to the enclosure and the additional enclosure.

6. The system of claim 1, further comprising a plurality of additional enclosures, wherein each of the plurality of additional enclosures comprises:
an additional radiation source configured to emit additional UV radiation towards an additional targeted area;
an additional holding area located within the additional targeted area, wherein the additional holding area is configured to receive the additional UV radiation;
one or more additional sensor devices comprising a sensor configured to detect the additional UV radiation and configured to record additional intensity information associated with the additional UV radiation; and
a controller configured to communicate with each of the additional radiation source and the one or more additional sensor devices of the plurality of enclosures, wherein the controller is configured to control and maintain an aggregate value of each of the intensity information and the additional intensity information above a predetermined value by adjusting a time period of the targeted area being exposed to the UV radiation and/or the additional UV radiation of each of the plurality of additional enclosures.

7. The system of claim 1, further comprising:
one or more additional radiation sources configured to emit the UV radiation, wherein the one or more additional radiation sources are positioned such that a distance between any adjacent two of the one or more additional radiation sources is at least 60% of a length of a diameter of the enclosure;
one or more additional sensor devices positioned opposing the one or more additional radiation sources within the targeted area, the one or more additional sensor devices configured to record the intensity information associated with the UV radiation; and
a controller configured to communicate with each of the additional radiation sources and the one or more additional sensor devices, wherein the controller is configured to control and maintain an aggregate value of the intensity information for each of the one or more sensor devices and the one or more additional sensor devices to be above a predetermined value.

8. A system, comprising:
an enclosure;
a radiation source configured to emit ultraviolet (UV) radiation within the enclosure towards a targeted area;
a holding area located within the targeted area configured to receive the UV radiation; and
one or more sensor devices, the one or more sensor devices comprising a first sensor configured to detect the UV radiation having a first filter profile and a second sensor configured to detect the UV radiation having a second filter profile, wherein the second filter profile allows a wider band of UV radiation to pass through as compared to the first filter profile;
wherein the one or more sensor devices are configured to record intensity information associated with the UV radiation, and wherein a location of one of the radiation source and the holding area is fixed within the enclosure.

9. The sensing device of claim 8, wherein the first filter profile and the second filter profile have an overlapping wavelength band within a UV wavelength range of 200 nanometers (nm) and 400 nm or within a UV wavelength range of 300 nm and 400 nm.

10. A sensing device, comprising:
a housing configured to receive an external radiation from a first direction;
a first sensor configured to detect an ultraviolet (UV) radiation having a first filter profile;
a first UV filter positioned adjacent to the first sensor within the housing such that a first radiation from the first direction received by the first sensor is configured to pass through the first UV filter;
a second sensor configured to detect the UV radiation having a second filter profile, wherein the second filter profile allows a wider band of UV radiation to pass through as compared to the first filter profile;
a second UV filter positioned adjacent to the second sensor within the housing such that a second radiation from the first direction received by the second sensor is configured to pass through the second UV filter; and
a controller, wherein the controller is configured to generate and output in accordance with the first radiation received at the first sensor and the second radiation received at the second sensor.

11. The sensing device of claim 10, wherein the first UV filter associated with the first filter profile is configured to pass through radiation with wavelengths between 300 nanometers (nm) and 400 nm, and wherein the second UV filter associated with the second filter profile is configured to pass through radiation with wavelengths between 220 nanometers (nm) and 400 nm.

12. The sensing device of claim 10, wherein the first filter profile and the second filter profile have an overlapping wavelength band within a UV wavelength range of 200 nanometers (nm) and 400 nm.

13. The sensing device of claim 12, further comprising an arithmetic unit configured to perform either a subtraction or an additional function to outputs of the first sensor and the second sensor.

14. The sensing device of claim 12, further comprising a control block configured to provide an indication that a wavelength of the UV radiation is inside the overlapping wavelength band.

15. The sensing device of claim 12, further comprising a control block configured to provide an indication that a wavelength of the UV radiation is outside the overlapping wavelength band.

16. The sensing device of claim 10, wherein the first filter and the second filter allow radiations of infra-red bandwidths to pass through.

17. The sensing device of claim 10, wherein the enclosure includes an opto-semiconductor package.

18. The sensing device of claim 17, wherein the first sensor includes a first die, the second sensor includes a second die, and the controller includes a third die, and wherein the first die, the second die, and the third die are located within the opto-semiconductor package.

19. The sensing device of claim 10, further comprising a semiconductor chip, wherein the first sensor includes a first photo-sensing area of the semiconductor chip and the second sensor includes a second photo-sensing area of the semiconductor chip.

20. The sensing device of claim 19, wherein the controller includes a logic circuit area of the semiconductor chip.
